(19) United States
(12) Patent Application Publication
(10) Pub. No.: US 2022/0411463 A1
(43) Pub. Date: Dec. 29, 2022

(54) MATE AND SEPARATE: A CONVENIENT AND GENERAL METHOD FOR THE SEPARATION AND PURIFICATION OF TARGET MOLECULES FROM BIOLOGICAL MEDIA BY PHASE TRANSITION OF PEGYLATED RECOGNITION AGENTS.

(71) Applicant: Khashayar KARIMIAN, Tehran (IR)

(72) Inventors: Khashayar KARIMIAN, Tehran (IR); Javad MOKHTARI, Tehran (IR); Amir Hassan ZARNANI, Tehran (IR); Aliakbar MOOSAVI-MOVAHEDI, Tehran (IR); Maryam NOURISEFAT, Bushehr (IR); Bita ZAMIRI, Tehran (IR); Leila FOTOUHI, Tehran (IR)

(21) Appl. No.: 17/432,929

(22) PCT Filed: Apr. 7, 2019
(86) PCT No.: PCT/IB2019/052851

§ 371 Date: Aug. 22, 2021

(30) Foreign Application Priority Data
Mar. 18, 2019 (IR) 13975014000301104

(51) Int. Cl. C07K 1/14; C07K 16/06; C07K 14/765; C12N 15/10

(52) U.S. Cl. CPC C07K 1/145; C07K 16/065; C07K 14/765; C12N 15/1006

(57) ABSTRACT

Biological small molecules, proteins or nucleic acids (target molecules, TM) are isolated in from biological media such as blood serum, cytoplasm, nucleoplasm etc. by a novel process (mate and separate) involving the use of PEGylated recognition molecule (PEG-RM) with high specificity and binding for TM, affording a macromolecular complex PEG-RM.TM, from which the target protein can be obtained in pure form.

### BACKGROUND OF THE INVENTION

Proteins play a vast array of structural and functional roles as catalyst, regulators in signal transductions, hormones, cell surface receptors, active transporters, drug carriers, antigens, antibodies, gene activators, repressors, and markers, etc. Separation and purification of a protein is a prerequisite to establishing its structure and function. Proteins also have ever-increasing commercial potentials. In the pharmaceutical industry alone this includes the production of biopharmaceuticals such as monoclonal antibodies (mAbs) [2], various diagnostic kits [3], personalized medicine in the identification of cell receptors and chimeric antigens in CAR T) [4-7] and gene editing (CRISP R) [8, 9], targeted drug delivery [10], isolation and purification of gene repressors [11], activators [12] and markers [13], etc.

In the past 30 years, the ever increasing medical applications of proteins have resulted in extensive and intensive research for their isolation and purification in both the academia and the pharmaceutical industry. The value of the biopharmaceutical market in 2016 was $228 billion and has been estimated to reach $460 billion by 2025 [14-16]. As of May 2018 the FDA had approved 80 mAbs as therapeutic agents [17] and in 2018 the FDA approved 17 new biological license applications [18, 19].

Nucleic acids contain the total information of a biological system and pay the central role of protein biosynthesis. Great advances in nucleic acid chemistry and molecular biology has paved the road for nucleic acid to play a paramount role in the next two decades as biopharmaceuticals (e.g. CAR T, CRISP R, gene editing, cell therapy, etc.) [8-9].

In downstream processes used for the separation and purification of mAbs a solution of protein become into physical contact with a neutral matrix with predefined physical properties (membrane filtration, microfiltration, ultrafiltration, diafiltration, size exclusion chromatography) or a matrix with predefined chemical properties (affinity chromatography, ion exchange chromatography, hydrophobic chromatography) [20-26]. The matrices used include, polysaccharides, ceramic and mixed mode materials [27], silica [28], fused core and magnetic particles [29-31]. Accordingly, isolation and purification of biological molecules are heterogeneous processes that are based on size, surface characteristics, charge or bio-properties of the target biomolecule. They are cumbersome, expensive and impose high burdens on the environment. Furthermore, they must be designed to minimize deleterious effect such as protein unfolding and aggregation. These process can result in physical or chemical stress downstream steps, which in the case of mAbs can be as many as 14 steps (vide supra). The multiplicity of the steps in current heterogeneous methods provides ample opportunity for protein aggregate formation or folding, posing a considerable challenge in mAb production on industrial scale [31-35]. mAb aggregates are immunogenic and can cause anaphylactic response in patients and even death. Accordingly, “re-purification” of a mAb may be necessary to obtain aggregate-free pharmaceutical mAb (usually size exclusion chromatography).

Other inherent problems with the use of heterogeneous chromatographic processes for protein isolation in large scale applications include ligand leakage of the affinity entity (e.g. staphylococci protein A or protein G) [20], back pressure, fouling, and ligand occlusion due to resin damage as well as channeling [36]. Also, some impurities retained by the affinity columns may co-elude with the product in the final stage of isolation [37, 38]. There is also a limitation on the quantity of protein mixtures that can be applied to a column.

The major difference between protein and nucleic acid isolation and purification for commercial use is quantitative in that proteins must be purified in kilogram to ton quantities but nucleic acids can be isolated in minute amounts in the laboratory and replicated with polymerases for which numerous methods are available. Nonetheless, isolation and purification of nucleic acids is also time consuming and expensive and utmost care must be exercised to prevent their degradation (e.g. by ubiquitous RNase). Numerous kits are available for nucleic acid isolation and purification. Common processes include guanidinium thiocyanate-phenol chloroform extraction, alkaline extraction methods, ethidium bromide-cesium chloride gradient centrifugation, silica matrix or poly (A)+RNA by OdTₓ-cellulose and anion exchange chromatography, solid phase extraction, using glass particles, diatomaceous earth, magnetic beads, Combined, these shortcomings in downstream heterogeneous processes for the isolation and purification of biological molecules increase production costs of mAbs, nucleic acids and small biological molecules, adversely affecting their affordability as therapeutic agents. For example, depending on the mAb titer, the downstream cost of production of mAbs has been estimated to be 45% (low titer, 0.1 g/l) to 80% (high titer. 1.0 g/l) of the total production cost of mAbs [20, 39]. Finally, the use of large volumes of buffers and various material (membranes, resins, etc.) with no or limited possibility of recycling impose considerable environmental burden.

The ever expanding application of mAbs as therapeutic agents in the treatment of various diseases from atopic eczema [40] and cancer [41] to potential non-addictive treatment of addiction to narcotics [42-44] is indicative of considerable future growth in protein biopharmaceuticals. Nonetheless, and in spite of many years of extensive and intensive research, the production of mAbs, in which protein A affinity chromatography constitutes the major cost center, remains to be very expensive [20, 39]. However, until now reported procedures have been focused on improvements of the existing heterogeneous processes used for the isolation of mAbs. [46-49]. For example, a protein A affinity resin has been reported to be recycled 300 times in the laboratory with proper washing and maintenance [45], this remains to be applicable to industrial scale. Yet, chromatographic methods are necessary for the production of pharmaceutical grade mAbs. But until now reported procedures have been focused on improvements of the existing heterogeneous processes used for the isolation of mAbs. [46-49].

There is therefore a need for an innovative and general procedure for the separation and purification of proteins that is devoid of the above-said problems of heterogeneous separation processes and does not involve so many steps. A homogeneous system with much reduced number of steps would certainly decrease production cost of mAbs and increase their affordability. In this report we describe a novel homogeneous procedure, coined mate and separate, for the separation and purification of proteins. The method is simple, convenient, and inexpensive and involves a few steps. We also discuss the applications of the mate and separate concept to the isolation of nucleic acids.

### BRIEF DESCRIPTION OF THE INVENTION

In the process divulged herein a recognition molecule (RM) with high specificity and binding for a target molecule (TM=protein, nucleic acid, small molecule) is covalently attached to a high molecular weight PEG to form a recognition macromolecule (PEG-RM). Addition of PEG-RM to a biological medium such as blood serum, cell cytoplasm, nucleoplasm, etc. results in the formation of a soluble macromolecular complex composed of PEG-RM and the target molecule (PEG-RM.TM). High specificity and binding of RM for TM is a result of the fundamental parameters in absorption of two molecules to each other, namely electrostatic (ionic and H-bonding), van der Waals (dipole-dipole), hydrophobic and to a lesser extent π interaction between PEG-RM and TM. Since PEG-RM.TM is endowed with the unique physicochemical properties PEG, the addition of a small quantity of a salt such as ammonium sulfate results in salt-assisted phase transformation of PEG-RM.TM from solution to a semisolid which can be conveniently separated from the biological solution by centrifugation or filtration. PEG-RM.TM is then dissociated to its constituents (PEG-RM+TM) by increasing the ionic strength or decreasing the pH of the solution which interfere with electrostatic (ionic and H-bonding), van der Waals (dipole-dipole), hydrophobic and to a lesser extent π interaction between PEG-RM and TM. Due to the presence of PEG in PEG-RM it can be separated from the solution by salt-assisted phase transformation by the addition of a small quantity of a salt such as ammonium sulfate. This results in the precipitation of PEG-RM as a semisolid which can be separated from TM by centrifugation of filtration, leaving pure TM in solution which can be isolated as solids with classical methods such as diafiltration or dialysis of salts followed by lyopholization of TM. The process of mate and separate is summarized in Scheme 1.

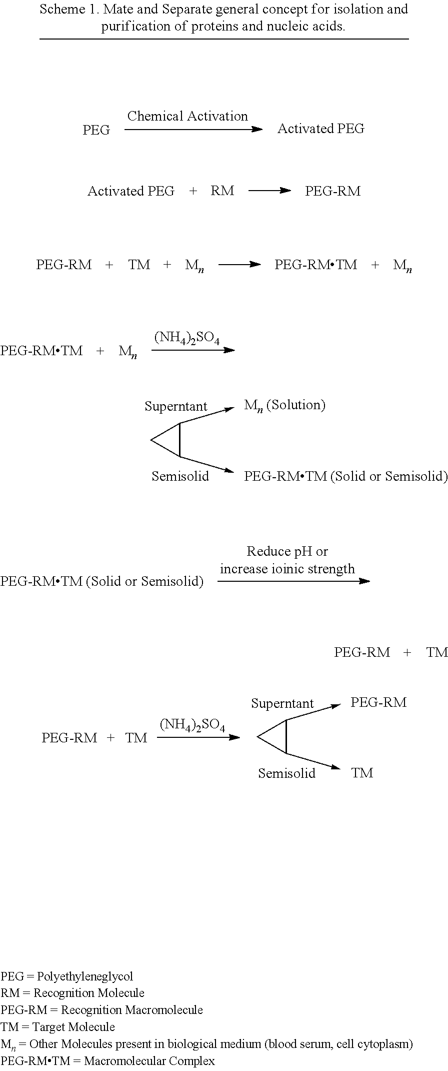

### SUMMARY OF THE INVENTION

In the present invention a very high molecular weight polyethylene glycol (PEG), protected at one end and activated at the other, is covalently attached to a recognition molecule (RM) which can be a protein, a nucleic acid or a small molecule. The RM is chosen for its high affinity and binding specificity for a target molecule (TM) which can be a protein or nucleic acid or a small molecule. This affords a recognition macromolecule (PEG-RM) the addition of which to a biological media such as a blood serum, cell cytoplasm or nucleoplasm or mitochondrial matrix results in the formation of a macromolecular complex (PEG-RM.TM). Since the macromolecular complex is endowed with the unique physico-chemical properties of PEG, the addition of a small quantity of a salt such as ammonium sulfate results in salt-assisted phase transition of the macromolecular complex (PEG-RM.TM) from solution to a solid or semisolid that can be separated from the biological medium by filtration or centrifugation. The separated macromolecular complex is then dissolved in a buffer and dissociated to its components (PEG-RM+TM) by decreasing the pH or increasing the ionic strength of the buffer. Addition of a small quantity of ammonium sulfate results in the phase transition of PEG-RM to a solid or semisolid that can be separated from the biological medium by filtration or centrifugation. This leaves the pure target molecule (protein or nucleic acid, small molecule) in solution (see Scheme 1 above).

### BRIEF DESCRIPTION OF THE FIGURES

FIG. 1 is a gel electrophoresis for the separation and isolation of pure Igs as TM from blood serum.

FIG. 2 is a gel electrophoresis for the separation and isolation of Igs from blood serum.

FIG. 3 is a gel electrophoresis for the separation and isolation of pure serum albumin as TM from blood serum.

FIG. 4 is an HPLC for the separation and isolation salicylic acid as TM.

FIG. 5 is an agarose gel electrophoresis for the separation and isolation of pure m-RNA as TM from white blood cells.

FIG. 6 is a schematic presentation of ammonium sulfate concentration-dependent salting-in of protein and slating-out of macromolecular complex.

FIGS. 7 and 8 show Dynamic Light Scattering of human serum albumin isolated by the mate and separate process.

### DETAILED DESCRIPTION OF THE INVENTION

The separate and separate concept is based on 3 fundamental chemical principles:

First, biological target molecules (TM=protein, nucleic acid, small molecule with biological activity) are very likely to have a recognition molecule (RM) to which they are adsorbed with high specificity and binding.

Second, high specificity and binding of RM for TM is a result of electrostatic (ionic and H-bonding), van der Waals (dipole-dipole), hydrophobic and to a lesser extent π interaction between PEG-RM and TM. These are fundamental forces in binding (mating) of two molecules in chemistry and are therefore the mate and separate concept is applicable to most biological molecules.

Third, PEG has very unique physico-chemical properties and while it is very soluble in water, it undergoes phase transition with the addition of small quantity of a salt such as ammonium sulfate, separating as a solid or a semi-solid from aqueous solutions.

In light of the above, PEGylation of the recognition molecule (RM) affords a recognition macromolecule (PEG-RM) that will attach to its target molecule (TM) with high specificity and binding (mate) to form a macromolecular complex (PEG-RM.TM). Due to the presence of PEG, PEG-RM.TM can be easily separated from the aqueous biological medium by the addition of ammonium sulfate (separate). Separation occurs because the macromolecular complex (PEG-RM.TM) is endowed with the unique physico-chemical properties of PEG. The macromolecular complex is then isolated by filtration or centrifugation. It is then dissolved in a buffer and dissociated to its components (PEG-RM+TM) by decreasing the pH or increasing the ionic strength or the temperature of the solution which disrupts the fundamental parameters involved in absorption of two molecules to each other, namely electrostatic (ionic and H-bonding), van der Waals (dipole-dipole), hydrophobic and to a lesser extent π interaction between PEG-RM and TM. Being endowed with the unique physico-chemical properties of PEG, the recognition macromolecule (PEG-RM) can be separated from the solution as a semisolid by the addition of a salt such as ammonium sulfate. Separation of the recognition macromolecule leaves the target molecule in solution (Scheme 1).

Proof of principle for the mate and separate concept was obtained using proteins as RM and TM, small molecules as RM and TM and nucleic acids as RM and TM to afford 9 different permutations. To this end, RMs were attached to PEGs of various molecular weight using PEGylation reactions shown in Scheme 2-4.

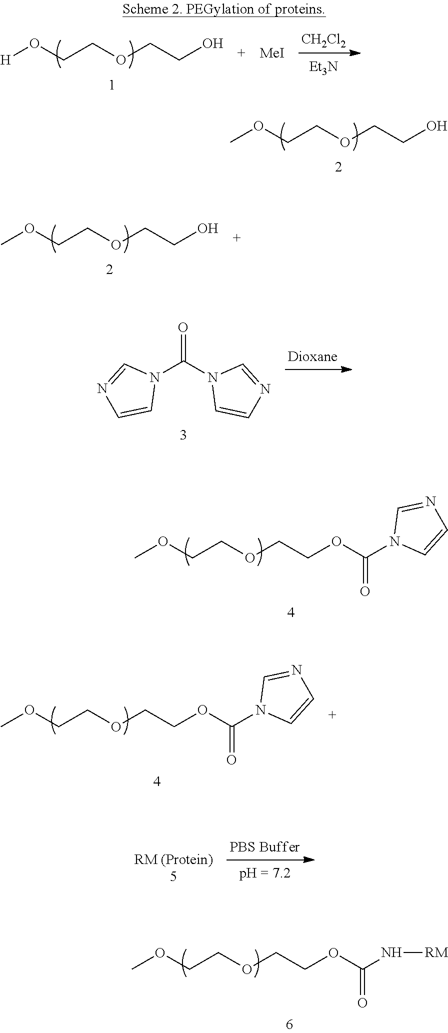

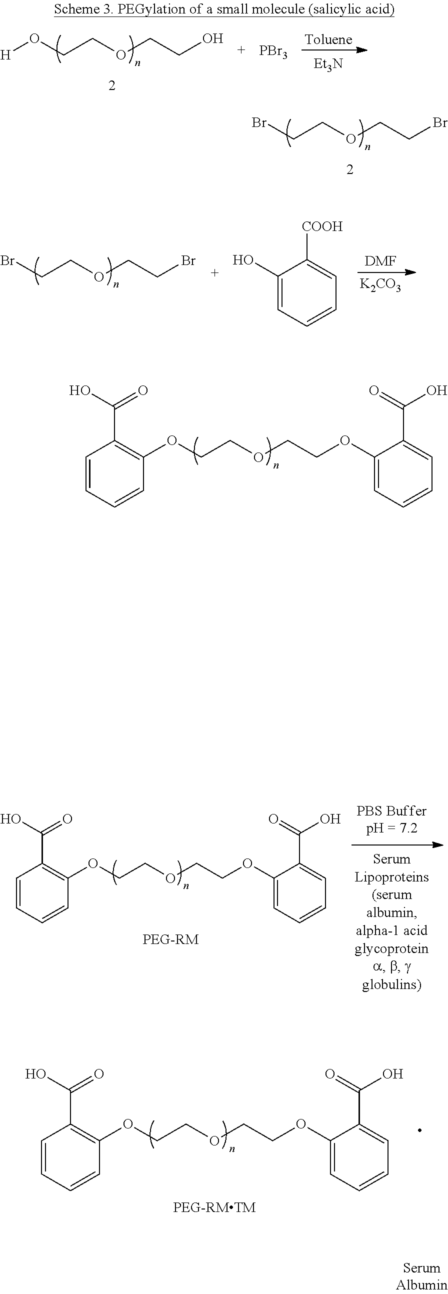

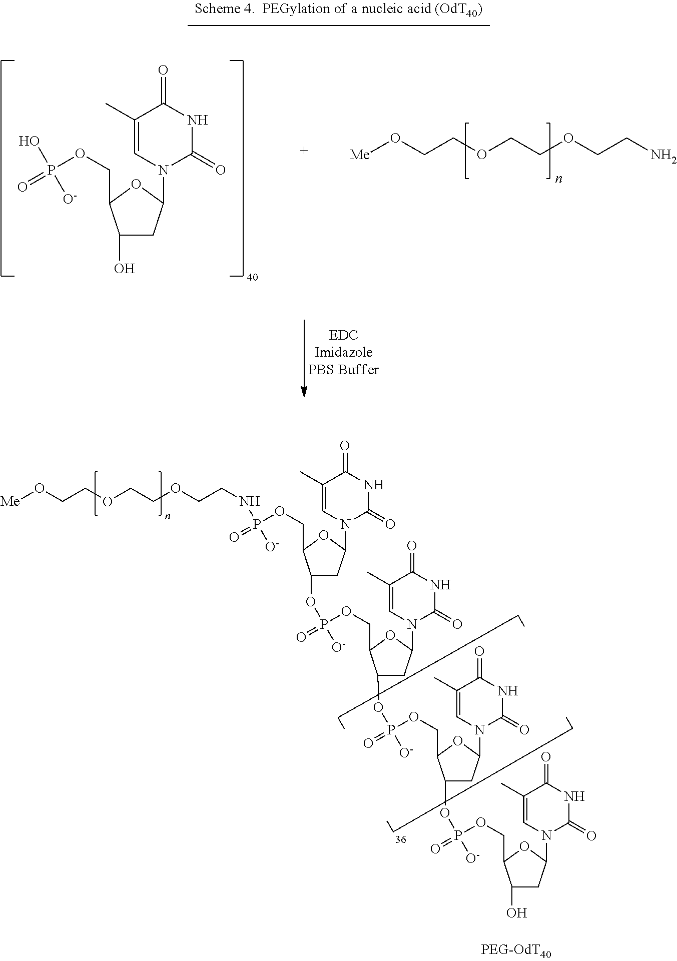

The mate and separate concept was then validated using proteins as RMs and as TMs as shown on Scheme 5.

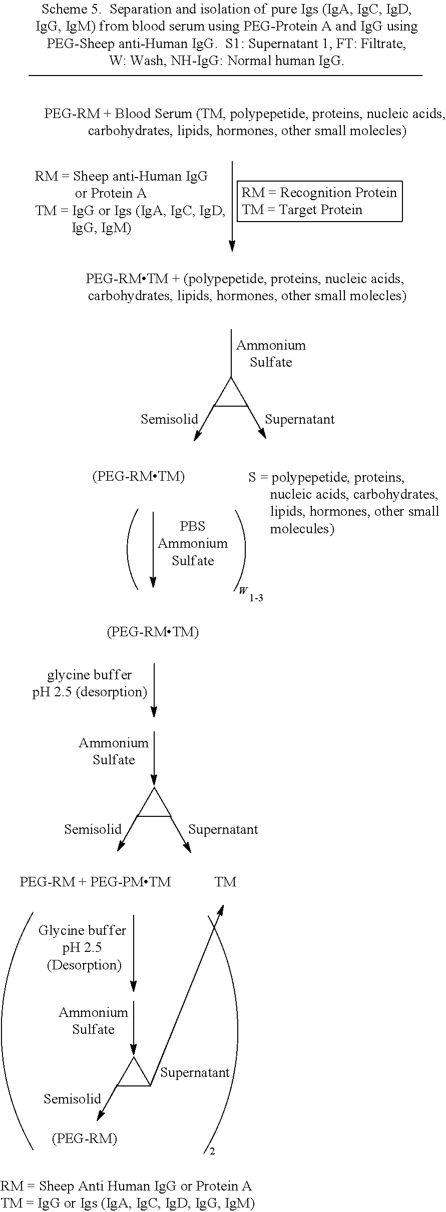

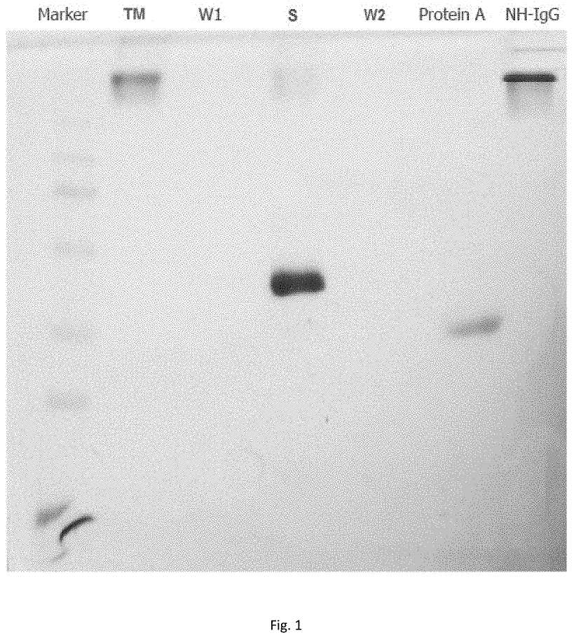

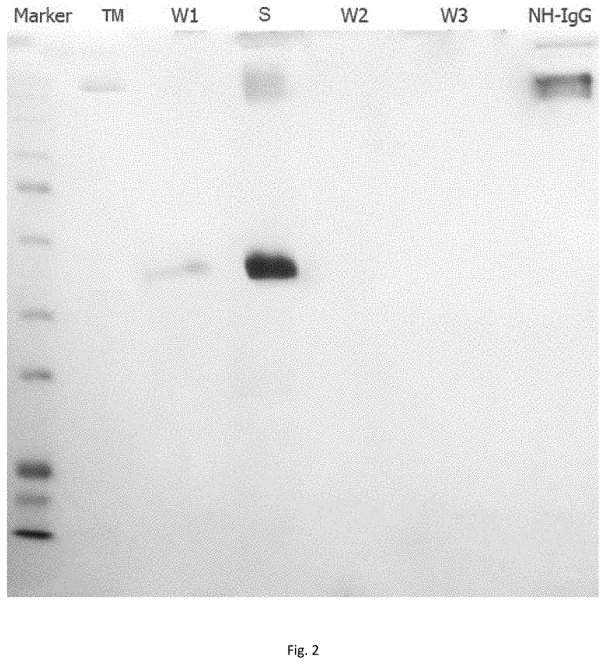

PEGylated Protein A was used for the isolation and purification of Igs (IgA, IgC, IgD, IgG, IgM) and sheep anti-human IgG was used for the isolation and purification of IgG. Results are provided in FIGS. 1 and 2 (SDS Page gel electrophoresis) and validate the application of the mate and separate concept in the separation and isolation of pure immunoglobulins using PEGylated Protein A or sheep antihuman IgG.

FIG. 1 is an SDS Page gel electrophoresis for the separation and isolation of pure Igs (IgA, IgC, IgD, IgG, IgM) as TM from blood serum using PEGylated protein A (PEG-Protein A)

FIG. 2 is an SDS Page gel electrophoresis for the separation and isolation of IgG as TM from blood serum using PEGylated sheep anti-human IgG (PEG-sheep anti-human IgG) The mate and separate concept was also validated using a PEGylated small molecule to isolate and purify a protein. To this end, salicylic acid was PEGylated using its hydroxyl group. The resulting recognition macromolecule (PEG-Salicylic acid) should bind with high affinity and specificity to serum albumin since drugs with carboxylic acid function are known to bind preferentially to serum albumin rather than other lipoproteins such as alpha-1 acid glycoprotein, or α, β, γ globulins. Accordingly, the process of PEGylation of salicylic acid was provided above (Scheme 6).

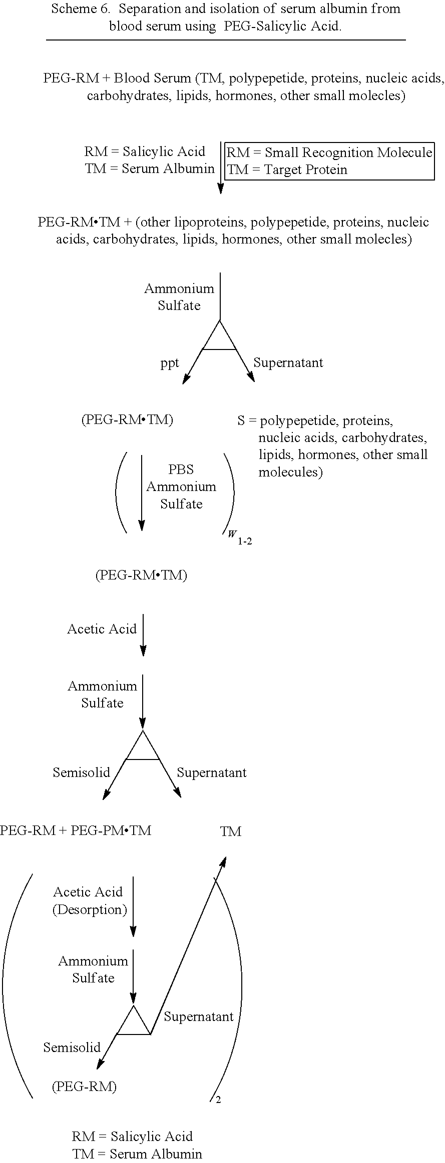

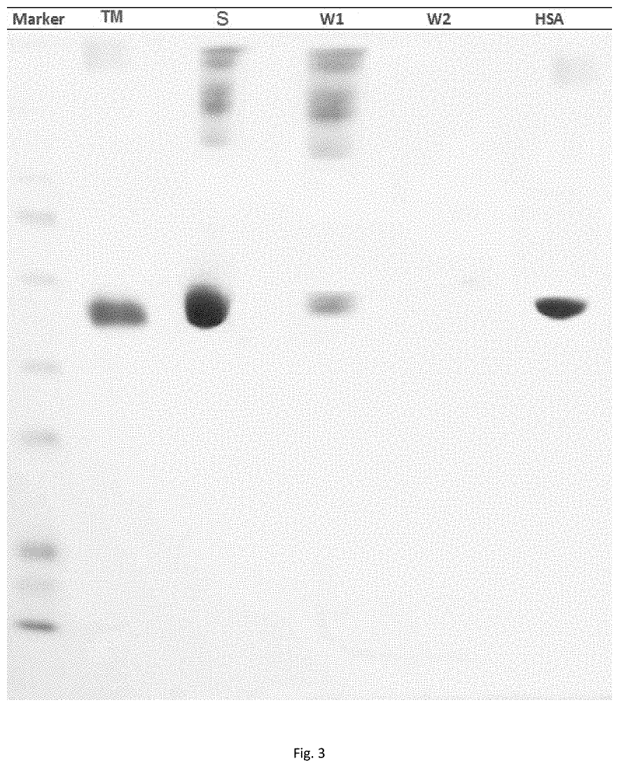

Results are shown in FIG. 3 and demonstrate complete preference for serum albumin (as compared to other lipoproteins such as alpha-1 acid glycoprotein, or α, β, γ globulins) and validate the application of the mate and separate concept to the separation and isolation of pure serum albumin using a PEGylated small RM.

FIG. 3 is an SDS Page gel electrophoresis for the separation and isolation of pure serum albumin as TM from blood serum using PEGylated salicylic acid (PEG-salicylic acid The possibility of interchangeability of RM and TM was demonstrated by using human serum albumin as RM to isolate salicylic acid in an equimolar mixture of 3 salicylic acid, capecitabine and deferiprone as shown in Scheme 7. The commonality of the fundamental forces that result in high specificity and binding (i.e. electrostatic, van der Waals, hydrophobic and to a lesser extent it interaction) is expected to result in preferential separation and isolation of salicylic acid (an acidic small molecule) as compared to capecitabine and deferiprone (neutral small molecules).

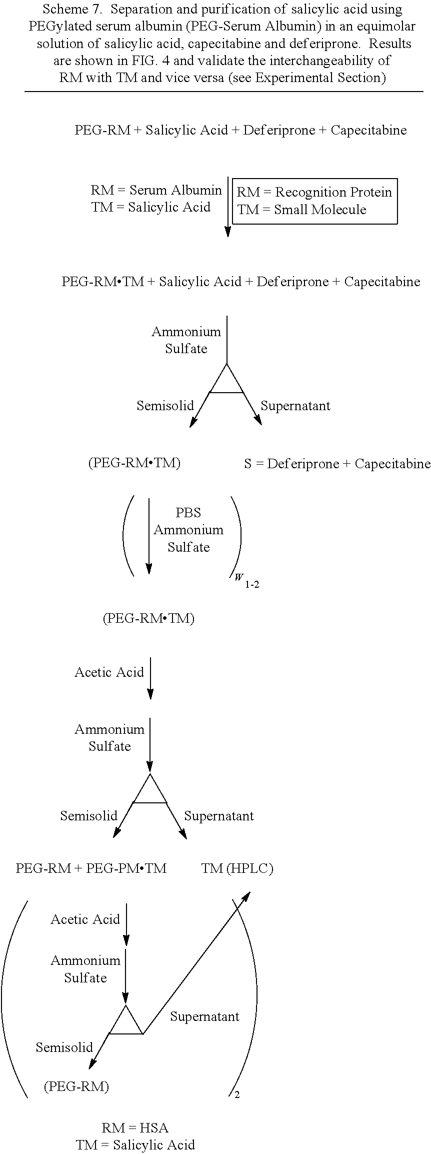

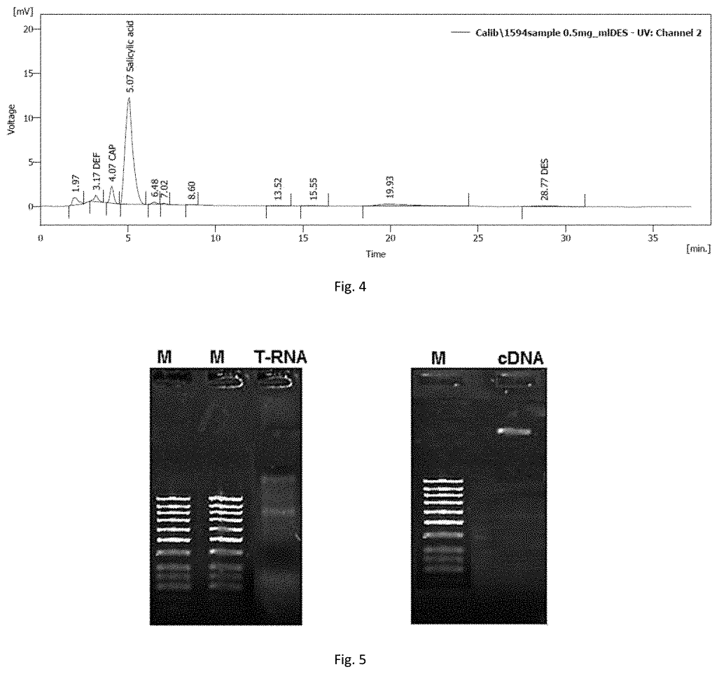

FIG. 4 is an HPLC for the separation and isolation of salicylic acid as TM from an equimolar mixture of Salicylic acid, deferiprone, capecitabine using PEGylated serum albumin (PEG-Serum Albumin).

Proof of principle for the interchangeability of RM with TM and vice versa is summarized in Formulae 1 and 2. Where A and B can be either RM or TM and Mₙ represents the innumerable molecules that are present in a biological solution such as blood serum, cell cytoplasm, nucleoplasm, etc.

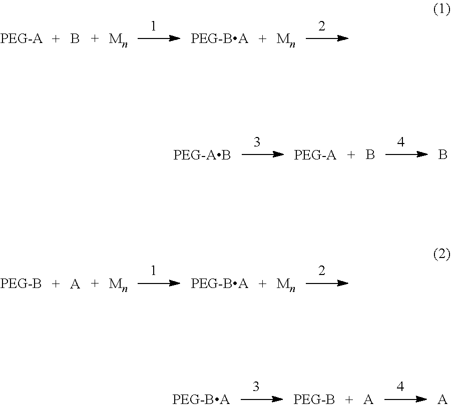

In Formulae 1 and 2 the steps are as follows:
1. Addition of PEGylated recognition molecule to biological medium containing the target molecule (A) as well as innumerable other molecules (Mₙ)
2. Formation of macromolecular complex, its phase transition to a semisolid by the addition of salt and separation of the macromolecular complex by filtration or centrifugation
3. Dissociation of the isolated macromolecular complex to recognition macromolecule and target molecule
4. Phase transition of recognition macromolecule to a semisolid by the addition of salt to a semisolid and its separation by filtration or centrifugation, leaving the pure target molecule in solution

Interchangeability of A and B and vice versa is due to the commonality of the fundamental forces that result in high specificity and binding (i.e. electrostatic, van der Waals, hydrophobic and to a lesser extent π interaction). Perhaps the simplest example is the binding of an antibody (RM) to an antigen (TM) or the binding of an antigen (RM) to an antibody (TM).

Finally, the mate and separate concept was validated using 2 nucleic acid as RMs and as TMs as shown on Scheme 8.

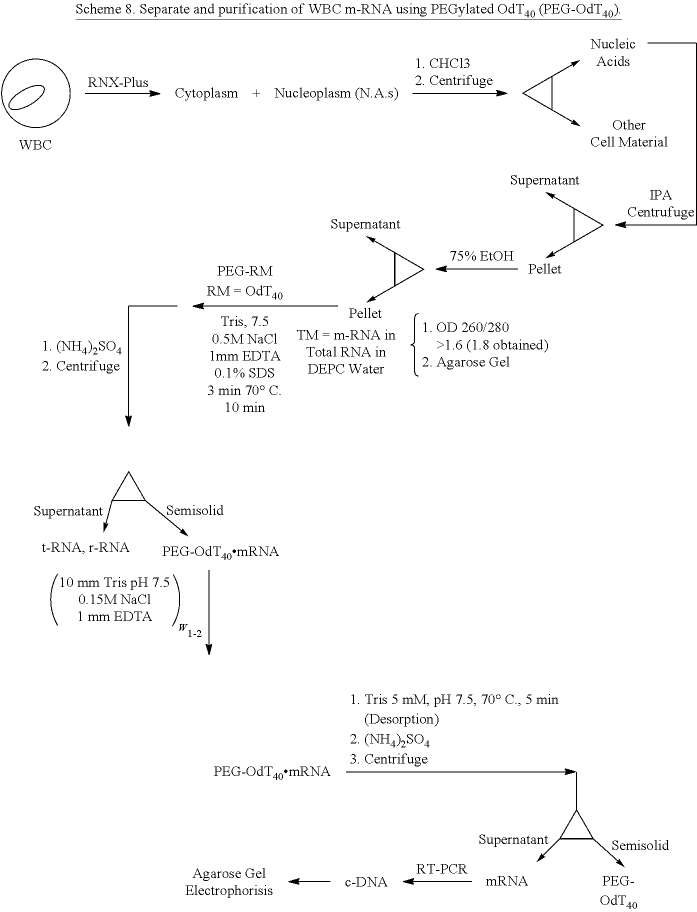

Results are shown in FIG. 5 and validate the application of the mate and separate concept to the separation and isolation of pure nucleic acids.

FIG. 5 is an agarose gel electrophoresis for the separation and isolation of pure m-RNA as TM from white blood cells using PEGylated OdT₄₀ (PEG-OdT₄₀). Left: Agarose Gel of extracted total RNA, Right: Agarose Gel of m-RNA expressed as complimentary DNA by polymerase chain reaction.

(see Experimental Section)

It should be noted that only m-RNA is capable of producing c-DNA by PCR. The formation of c-DNA shown in FIG. 5, Right, therefore validates the mate and separate concept in the isolation and purification of nucleic acids.

The mate and separate processes for the isolation and purification of proteins, nucleic acids and small molecules has a number of other advantages. With regard to proteins, we used a very high molecular weight PEG for the PEGylation of the recognition molecule RM (especially when RM=protein) for the following reasons:

First, to prevent multi-PEGylation since this involves heteroatoms such as SH, NH and OH functional groups of the peptide which are also involved in the recognition of the TM by electrostatic (ionic and H-bonding), van der Waals (dipole-dipole), hydrophobic and to a lesser extent π interactions [50].

Second, to ensure dominance of the precipitative character of PEG over the physicochemical properties of the macromolecular complex (PEG-RM.RM), which guarantees phase transition of (PEG-RM.RM) to a semisolid at low salt concentration, concomitant with increased solubility of unbounded proteins.

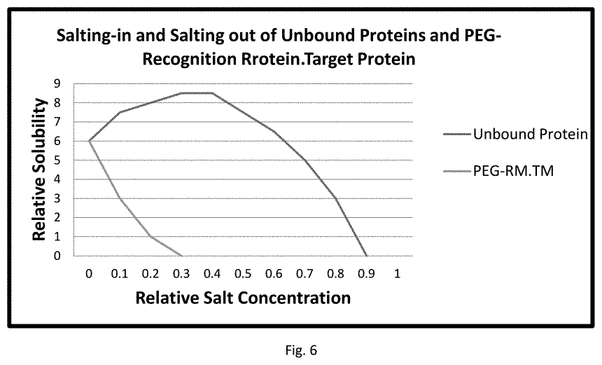

Third, the use of a low salt concentration, in addition to increasing the solubility of proteins, is known to decrease nonspecific protein-protein interaction, thereby providing a better chance for high specificity and binding of PEG-RM to the target protein (TM) [51, 52] In light of the above, the salting-in of unbounded proteins and salting out of PEG-RM.TM as a function of salt concentration should follow the schematic representation in FIG. 6.

FIG. 6 is a Schematic presentation of ammonium sulfate concentration-dependent salting-in of proteins and slating-out of macromolecular complex (PEG-RM-TM) using high molecular weight PEG.

In fact, salting-in of unbounded proteins at low salt concentration concomitant with salting-out of high molecular weight PEG is a fortuitous coincidence that provides an ideal condition for salt-assisted phase transition of PEG-RM.TM.

Furthermore, it is important to note that in the absence of ammonium sulfate mating of PEG-RM with TM to afford PEG-RM.TM is an equilibrium phenomenon and according to the Le Chatelier Principle the addition of salt shifts the equilibrium to the formation of the PEG-RM.TM as it departs from the aqueous medium in the form of semisolid (salt-assisted phase transition) [53]. It is also important to note that protein isolation by the mate and separate concept does not cause leaching of recognition protein (e.g. protein A or sheep anti-human IgG). This can be seen in FIGS. 1 and 2 respectively in which the samples are devoid of protein A or sheep anti-human IgG.

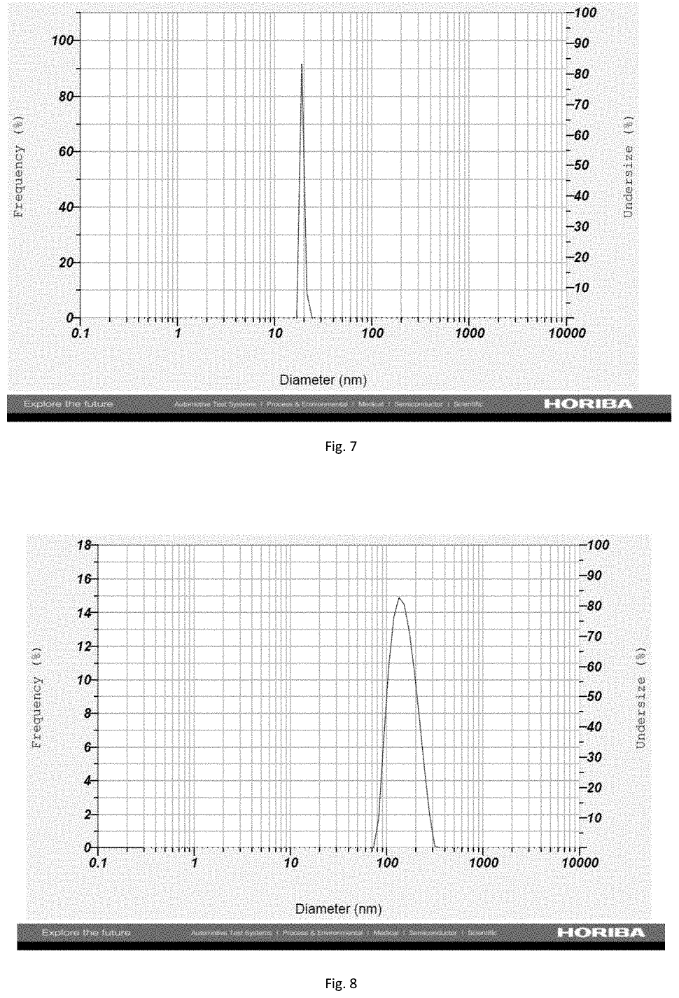

Equally as important as the above-mentioned advantages is the fact that the proteins isolated and purified from the mate and separate process are devoid of aggregates. As indicated above, aggregate formation is a major challenge in mAb production, especially in view of the fact that such protein entities with immunogenic properties can form in both the upstream and the downstream production of mAbs as well as their formulation, transport and storage [54, 55]. Our initial results based on SDS PAGE (see FIGS. 1, 2 and 3) demonstrate that Igs, IgG and HSA isolated by the mate and separate concept are devoid of aggregates [56]. Also, as shown in FIG. 7, Dynamic Light Scattering (DLS) experiment shows the hydrodynamic diameter of freshly isolated human serum albumin isolated by our method to be 18 nm, whereas DLS of the same protein kept at −20° C. for 12 months shows a hydrodynamic diameter of 144 nm (FIGS. 7 and 8) [57], demonstrating the presence of aggregates (it is well-established that proteins form aggregates as a function of time). The isolation of monomeric form of proteins in the mate and separate process could be a result of celerity of our process which afford to pure target protein in less than one hour. Conventional processes of mAb production can be as many as 14 steps with high residency time of the target protein in various steps of the process. Furthermore, our process does not exert undesirable chemical or physical stresses on the TM, which are known to cause protein aggregation. Moreover, high specificity and binding of PEG-RM to TM and comports with the contention that surface properties of aggregates are different from single protein to the extent that PEG-RM does not bind to aggregates, if in fact any aggregate is formed. Finally, the use of a very high molecular weight PEG could result in a formidable steric hindrance that would not allow binding of a very high molecular weight aggregate to PEG-RM.

FIG. 7 is a Dynamic Light Scattering of human serum albumin isolated by the mate and separate process.

FIG. 8 is a Dynamic Light Scattering of human serum albumin isolated by the mate and separate process and kept at −20° C. for 12 months.

As regards the isolation and purification of nucleic acids using the mate and separate process, the use of Ammonium sulfate for the precipitation of PEG-Nucleic Acid. Nucleic acid has been reported to stabilize m-RNA [58] by inhibiting various RNases. Inhibition is caused by competition of sulfate moiety of ammonium sulfate with the phosphate moiety of RNA [59]. In light of the simplicity of the process, purity of the isolated product (proteins, nucleic acids or small molecules) as well as interchangeability of recognition molecule with the target molecules and vice versa, the actual and potential utility of the mate and separate process are summarized in Table 1.

TABLE 1
Various permutation of the mate and separate concept

| No | (PEG-RM) | (Target Molecule = TM) | Examples of Utility |
|---|---|---|---|
| 1 | RM = Small Molecule | Protein | See Example 7 |
| | (Salicylic Acid) | (Human Serum Albumin) | |
| 2 | RM = Small Molecule | TM = Nucleic Acid | On-going work in our laboratories |
| | | | Bleomycin binding to a specific |
| | | | base sequence in nucleic acids |
| 3 | RM = Protein | TM = Small Molecule | See Example 2 |
| | (Human Serum | Salicylic Acid | |
| | Albumin) | | |
| 4 | RM = Protein | TM = Protein | See Examples 1 |
| | (Protein A or Sheep | (Igs or IgG) | |
| | Anti-human IgG) | | |
| 5 | RM = Protein | TM = Nucleic Acid | On-going work in our laboratories |
| | | | Isolation repressor or activator |
| | | | segments of genes |
| 6 | RM = Nucleic Acid | TM = Small Molecule | On-going work in our laboratories |
| | | | Daunomycin binding to a specific |
| | | | base sequence in nucleic acids |
| 7 | RM = Nucleic Acid | TM = Protein | On-going work in our laboratories |
| | | | Isolation repressor or activator |
| | | | segments of genes |
| 8 | RM = Nucleic Acid | TM = Nucleic Acid | Isolation of m-RNA using OdT₄₀ |

### Experimental

Material and Methods

All reagents including PEG-1,000,000 were obtained from commercially available sources such as Sigma-Aldrich, Fluka or Merck and were used without further purification. Human blood was obtained from the peripheral vein of healthy donor with sterilized disposable plastic syringe. The blood was preserved without anticoagulant in sterilized test tubes in vertical position until clot was formed. The clot was then removed from the test tube gently with a glass rod or a swab and the serum was centrifuged at 1000 g for 10 minutes.

1. Isolation of Igs or IgG Using PEGylated-Protein A or PEGylated-Sheep Anti-Human IgG

1.1. Monomethylation of PEG-1,000,000. To a solution of PEG-1,000,000 (50 g, 0.05 mmol) in 500 ml of dioxane was added freshly distilled triethylamine (7 μl, 0.05 mmol). Methyl iodide (3.1 μl, 0.05 mmol), dissolved in 20 ml of dry dioxane, was added dropwise over 1 h at 25° C. and the mixture was stirred for 24 h at room temperature. Diethyl ether (500 ml) was added slowly and the resulting white precipitate was filtered and washed with cooled dioxane and then diethyl ether. After drying to a constant weight, 49 g of product was obtained (97%).

1.2. Activation of MeO-PEG-OH by N,N-Carbonyl Diimidazole. To the solution of monomethyl PEG-1,000,000 (20 g, 0.02 mmol) in 250 cc dioxane was added N,N-carbonyl diimidazole (0.032 g, 0.2 mmol). The reaction mixture was heated to 60° C. for 24 h, cooled to room temperature and 250 cc diethyl ether was slowly added. The resulting white precipitate was filtered and washed with cooled dioxane and diethyl ether. After drying to a constant weight, 19 g of activated PEG was obtained (95%).

1.3. Coupling of Recognition Protein and activated PEG (RM=Sheep anti-human IgG or PEG-Protein A). To a solution of Sheep-Anti Human IgG (1 ml, 0.5 mg/ml, 3.3×10⁻⁶ mmol) or Protein A (1 ml, 0.5 mg/ml, 3.3×10⁻⁶ mmol) in 4 ml PBS buffer with pH=7.2 was added activated PEG (66 mg, 6.66×10⁻⁵ mmol). The resulting solution was gently shaken at 0-10° C. for 10 days.

Completion of the coupling was monitored by Native PAGE. A saturated solution of ammonium sulfate was then added to the reaction mixture. The semisolid material was separated by centrifugation. The semisolid was washed with saturated solution of ammonium sulfate (1 ml), and centrifuged again. The semisolid residue (PEG-Sheep anti-human IgG or PEG-Protein A) was used for the separation of IgG or Ig from the blood serum.

1.4. solation and Purification of IgG from Blood Serum with PEG-RM (RM=Sheep-anti human IgG or PEG-Protein A). General procedure for IgG purification using the synthesized PEG-RM and human serum: Blood was withdrawn and diluted with 1 ml PBS buffer pH 7.2. Blood cells were separated from plasma by centrifugation (500×g during 5 min). PEG-RM was then added to the diluted sample of human serum and gently shaken for 45 min at room temperature. A saturated solution of ammonium sulfate (0.5 ml) was added to the mixture which resulted in salt assisted phase transformation of PEG-RM.IgG to a semisolid in about 2 minutes. The semisolid was separated by centrifugation at 3000 RPM for 5 minutes. PBS buffer (0.5 ml) was added to the semisolid and solution was gently mixed to wash away unbound serum components, followed by centrifugation and decantation. The wash procedure was repeated two more times by the addition of ammonium sulfate, separation of the semisolid and the addition of Phosphate-buffered saline (PBS, 1 ml). The macromolecular complex PEG-RM. IgG was then dissociated to its components (PEG-RM+IgG) by the addition of an acidic buffer (e.g., 0.15 M glycine-HCl, pH 2.5). PEG-RM was removed as a semisolid by the addition of ammonium sulfate and centrifugation as discussed above. The final solution contained pure IgG or Ig and ammonium sulfate, which was removed by dialysis in 0.15 M PBS buffer, pH 7.2, twice over a 2-h at a ratio of 1 volume of sample to 100 volume of buffer. PEG-RM was recovered in its purified form by washing 3 times with 0.15 M PBS buffer, pH 7.2 and stored in the buffer containing 0.01% NaN₃.

2. Isolation of Pure Serum Albumin Using PEGylated-Salicylic Acid

2.1. Activation of OH-PEG-OH with Phosphorous Tribromide. The process of Bückmann et al. [60] was used with minor modification. PEG 1,000,000 (50 g, 0.05 mmol) was dissolved in 500 ml of toluene followed by distillation of 100 ml of the solvent to remove trace of moisture. After cooling to 35° C., freshly distilled anhydrous triethylamine (0.054 g, 0.54 mmol) was added. Phosphorus tribromide (0.146 g, 0.54 mmol), dissolved in 20 ml of dry toluene, and was then added dropwise over 1 h at 35° C. under a dry nitrogen atmosphere with continuous stirring. The mixture was refluxed for 1 h. Triethylammonium bromide byproduct was removed by passing the hot solution through a bed of Celite. The filtrate was stored at 4° C. overnight, affording activated PEG, which was filtered at 4° C. The solid product was further purified by dissolving in 2.5 l of absolute ethanol at 60° C. The ethanolic filtrate was stored overnight at 4° C. to recrystallize the product. The solid material was separated by filtration and washed with cold ethanol and then ether. After drying in a vacuum desiccator 49 g of a pale yellow product was obtained (98%).

2.2. Coupling of salicylic acid and activated PEG. To a solution of salicylic acid (1.0 g, 7.24 mmol) in 100 ml DMF was added potassium carbonate (2.0 g, 14.48 mmol), followed by activated PEG (10 g, 0.01 mmol). The resulting solution was heated to 80-90° C. for 10 hours. Reaction completion was monitored by TLC. The mixture was then cooled to ambient temperature and diethyl ether (200 ml) was added drop-wise with stirring. The resulting precipitate was filtered and washed with diethyl ether and dried to a constant weight (9.8 g, 98% Yield).

2.3. Isolation and Purification of Serum Albumin with PEGylated Salicylic Acid. Blood serum was obtained as above. PEG-salicylic acid was added to a diluted sample of human serum (10 μl) and gently shaken for 45 min at room temperature. A small quantity of a saturated solution of saturated ammonium sulfate (ca. 0.5 ml) was then added to the solution causing immediate formation of a PEG-Salicylic Acid.Serum Albumin as a semisolid, which was centrifuged at 4000 RMM for 5 minutes and decanted. The semisolid was washed twice with a saturated solution of ammonium sulfate (1 ml) to ensure removal of unbounded proteins and other potential impurity. The final semisolid was dissolved in 10% acetic acid to disrupt molecular interactions such as electrostatic, van der Waals, hydrophobic and to a lesser extent π interactions required for high affinity and specificity of RM for TM. The mixture was stirred for 10 minutes to cause dissociation of PEG-Salicylic Acid.Serum Albumin to PEG-Salicylic Acid+Serum Albumin. Addition of a small quantity of saturated solution of ammonium sulfate resulted in salt-assisted phase transition of PEG-Salicylic Acid to a semisolid, leaving pure serum albumin in solution. Ammonium sulfate was removed by dialysis in 0.15 M PBS buffer, pH 7.2, twice over a 2-h period at a ratio of 1 volume of sample to 100 volume of buffer. The recognition macromolecule PEG-RM was recovered in its purified form by washing 3 times with 0.15 M PBS buffer, pH 7.2 and stored in the buffer containing 0.01% NaN₃. It can be used in the next cycle of isolation and purification of serum albumin.

3. Isolation of Salicylic Acid from Mixture of Drugs Using PEGylated-Human Serum Albumin

3.1.Coupling of Human Serum Albumin and Activated PEG

The process of experiment 3.1 (coupling of protein A and activated PEG) was used.

3.2. Isolation and Purification of Salicylic Acid from an Equimolar Mixture of Drugs

Using PEGylated Human Serum Albumin. An equimolar mixture of salicylic acid (0.05 mg, 0.375 μmole), deferiprone (0.051 mg, 0.375 μmole), capecitabine (0.134 mg, 0.375 μmole) was prepared in 0.5 cc PBS buffer with pH 7.2. The PEGylated human serum albumin (375 mg, 0.375 μmole) was added to the drug mixture and the solution was gently shaken for 1 hour at room temperature. A small quantity of a saturated solution of saturated ammonium sulfate (ca. 0.5 ml) was then added to the solution causing immediate formation of a PEG-HSA. “Drug” as a semisolid, which was centrifuged at 4000 RMM for 5 minutes and decanted. The semisolid was washed twice with a saturated solution of ammonium sulfate (1 ml) to ensure removal of unbounded proteins and other potential impurity. The final semisolid was dissolved in 5% acetic acid to disrupt molecular interactions such as electrostatic, van der Waals, hydrophobic and to a lesser extent π interactions required for high affinity and specificity of RM for TM. The mixture was stirred for 10 minutes to cause dissociation of PEG-HSA. “Drug” to PEG-HAS+“Drug”. Addition of a small quantity of saturated solution of ammonium sulfate resulted in salt-assisted phase transition of PEG-HSA to a semisolid, leaving pure “Drug” in solution. The aqueous solution was evaporated under reduced pressure to dryness. To the solid mixture containing the “Drugs” was added ethyl acetate, filtered and the solvent was remove under reduced pressure to afford an oil which was subjected to HPLC chromatography which afford good separation of the drugs. Column: RP-C18, 300*4.0 mm, 10 micron: Mobile Phase: 550 ml 0.02 molar KH₂PO₄ buffer, pH 2.5 and 450 ml acetonitrile). The HPLC chromatogram showed 77% salicylic acid and 5% capecitabine and 3% deferiprone.

4. Isolation of WBC m-RNA Using PEGylated-OdT₄₀

4.1. Synthesis of MeO-PEG-NH₂. The process of Bückmann et al. [58] was used with minor modification. Monomethoxylated PEG 35,000 (20.0 g, 0.57 mmol) was dissolved in 500 ml dry toluene. After cooling to 35° C., freshly distilled anhydrous triethylamine (0.06 g, 0.6 mmol) was added. Phosphorus tribromide (0.162 g, 0.6 mmol), dissolved in 20 ml of dry toluene, and was then added dropwise over 1 h at 30° C. under a dry nitrogen atmosphere with continuous stirring. The mixture was refluxed for 1 h. Triethylammonium bromide byproduct was removed by passing the hot solution through a bed of Celite. The filtrate was stored at 4° C. overnight, affording activated PEG, which was filtered at 4° C. The solid product was further purified by dissolving in 100 ml of absolute ethanol at 60° C., treated with decolorizing charcoal, filtered and the ethanolic filtrate was stored overnight at 4° C. to recrystallize the product. The solid material was separated by filtration and washed with cold ethanol and then ether. After drying in a vacuum desiccator 18 g of a pale yellow product was obtained (90%). The halogenated PEG was added slowly to a 100 ml 15% ethanolic solution of ammonia (large excess) and refluxed for 24 hours. The solvent was removed under reduced pressure and residual ammonia was removed by the addition of 50 ml ethanol followed by its removal under reduced pressure. The resulting solid was uses in the next step.

4.2. Coupling of OdT₄₀ and activated PEG. A solution of 0.1 molar imidazole was prepared by the addition of 340 mg imidazole to 50 ml PBS buffer, containing 10 mM EDTA, pH 7.2. MeO-PEG-NH₂ (PEG 35,000, 8.75 g, 0.25 mmol) was added to the imidazole buffer solution, resulting in a 0.25 molar solution of MeO-PEG-NH₂. A 100 μl aliquot portion of this solution was added to a microtube (Solution A). In a separate microtube, 1.25 mg (8 μmole) EDCE (1-Ethyl-3-(3-dimethylaminopropyl)-carbodiimide was prepared in 20 μl of the PBS buffer followed by the addition of 252 μl of a 1000 μmolar solution of OdT₄₀ corresponding to 60 μg OdT₄₀(Solution B). Solution A (20 μl) was then added to Solution B and mixed well, followed by the addition of a second portion of solution A (80 μl). The mixture was gently shaken for 24 hour at 37° C. Agarose gel electrophoresis was used to ascertain reaction completion, at which point the mixture was passed through a small anion exchange resin (1.0 g) using the PBS buffer.

4.3 Separation and Isolation of pure m-RNA from White Blood Cells. To 1 ml human blood was added RBC Lysis buffer (10 ml) and the mixture was gently shaken for 10 minutes. The mixture was then centrifuged (4° C., 4000 rpm, 5 minutes). Red blood cells were removed by repeating the process. Total RNA was extracted using RNX-Plus from CinnaClone (1 ml) and gently vortexed for 5-10 seconds and incubated the mixture for 5 minutes at room temperature. To the resulting suspension was added chloroform (200 μl) and the mixture was gently shaken for 15 seconds and incubated on ice for 5 minutes, followed by centrifugation (4° C., 12,000 rpm, 15 minutes). The top layer containing total RNA was carefully transferred to a 1.5 ml microtube. An equivalent volume of isopropanol was added and the mixture gently shaken and incubated on ice for 15 minutes, followed by centrifugation (4° C., 12,000 rpm, 15 minutes). The process was repeated using 75% ethanol and the mixture was centrifuged (4° C., 7500 rpm, 15 minutes). The top layer was carefully removed and to the pellet was added DEPC water (50 μl) and the solution was examined for total RNA content by measuring OD ratio at 260/280 nm using a Nanodrop instrument (Thermo-Fischer) and agarose gel electrophoresis (FIG. 5, Left). The OD ratio was 1.8 (should be more than 1.6). Combined with agarose gel electrophoresis results, the presence of total RNA was confirmed. To the total RNA solution was added 10 mM Tris buffer, pH 7.5 (250 μl) containing 0.5 mM sodium chloride, 1 mM EDTA and 0.1% SDS. Subsequently, the solution of PEG-OdT₄₀ (250 μl) from Experiment 4.2 was added to the total RNA solution. The mixture was incubated at 70° C. for 3 minutes and then at for 10 minutes at room temperature. Ammonium sulfate (0.2 g, 1.5 mmol) was then added and the resulting semisolid (PEG-OdT₄₀.m-RNA) was separated by centrifugation (4° C., 4,000 rpm, 5 minutes). The separated semisolid was washed with 100 μl of 10 μM Tris buffer pH 7.5, containing 0.15 mM sodium chloride and 1 mM EDTA. Dissociation of PEG-OdT₄₀.m-RNA to its components (mRNA and PEG-OdT₄₀) was achieved by the addition of 500 μl preheated Tris buffer, 5 mM, pH 7.5 at 70° C. and incubating for 10 minutes. PEG-OdT₄₀ was converted to a semisolid by the addition of ammonium sulfate (0.2 g, 1.5 mmol). The semisolid was removed by centrifugation (4° C., 4,000 rpm, 5 minutes), leaving m-RNA in solution, which was used for polymerase chain reaction (PCR) using Easy c-DNA Transcription Kit from Parstous Biotechnology. C-DNA formation (FIG. 5, Right) demonstrates successful isolation and purification of nucleic acid by this process.

### REFERENCES

1. Hofmeister, F. Ueber die darstellung von krystallisirtem eieralbumin and die krystallisirbarkeit colloider stoffe. Zeitschrift für Physiologische Chemie 14, 165-172 (1890).
2. Singh, S., et al. Monoclonal antibodies: a review. Cur. Clin. Pharmaco. 13, 85-99 (2018).
3. World Health Organization https://www.who.int/diagnostics_laboratory/evaluations/161214_prequalified_product_list.pdf
4. Weiner, G. J. Building better monoclonal antibody-based therapeutics. Nature Reviews, Cancer 15, 361-370 (2015).
5. June, C. H., O'Connor, R. S., Kawalekar, 0. U., Ghassemi, S., Milone, M. C. CAR T cell immunotherapy for human cancer. Science 359, 1361-1365 (2018).
6. Maciejko, L., Smalley, M., Goldman, A. Cancer immunotherapy and personalized medicine: emerging technologies and biomarker-based approaches. J. Mol. Biomark. Diagn. 8, 1-5 (2017).
7. Cross, R. Controlling CAR-T: how scientists plan to make the engineered T cell therapy safer, and work for more cancers. Chemical and Engineering News, 96, Issue 19 (2018).
8. Adli, M. The CRISPR tool kit for genome editing and beyond. Nature Comm. 9, Article Number 1911 (2018).
9. Chira, S. et al. CRISPR/Cas9: Transcending the reality of genome editing. Molecular Therapy: Nucleic Acids 7, 213-222 (2017).
10. Rosenblum, D., Joshi, N., Tao, W., Karm, J. M., Peer, D. Progress and challenges towards targeted delivery of cancer therapeutics. Nature Comm. 9, Article Number 1410 (2018).
11. Schildkraut, I. Encyclopedia of Genetics, S. Brener and J. H. Miller Editors (Academic Press, NewYork, 2001, p 1678).
12. Dong, C., Fontana, J., Patel, A., Carothers, J. M. Zalatan, J. G. Synthetic CRISPR-Cas gene activators for transcriptional reprogramming in bacteria. Nature Comm. 9, Article Number 2489 (2018).
13. Grover, A., Sharma, P. C. Development and use of molecular markers: past and present. Critical Rev. Biotechnol. 36, 290-302 (2016).
14. Martin, K. Market and Market Access, 15th Biosimilar Medicines Conference (Medicines for Europe). London: Quintiles IMS (2017).
15. Moorkens, E. The Market of Biopharmaceutical Medicines: a snapshot of a Diverse Industrial Landscape. Front Pharmacol. 8, 314 (2017).
16. Global Biopharmaceutical Market Analysis & Trends—Industry Forecast to 2025 https://www.researchandmarkets.com/reports/4296419/global-biopharmaceutical-market-analysis-and.
17. Cai, H. H. et al. Therapeutic monoclonal antibodies approved by FDA in 2017. MOJ Immunol. 6, 82-84 (2018).
18. Urquhart, L. FDA new drug approvals in Q1 2018. Nat. Rev. Drug Dis. 17, 309 (2018).
19. https://www.fda.gov/BiologicsBloodVaccines/DevelopmentApprovalProcess/Biological ApprovalsbyYear/ucm596371.htm.
20. Sommerfeld, S., Strube, J. Challenges in biotechnology production—generic processes and process optimization for monoclonal antibodies. Chem. Enging. Process. 44, 1123-1137 (2005).
21. Kelley, B. Industrialization of mAb production technology, the bioprocessing industry at a crossroads. mAbs. 1, 443-452 (2009).
22. Binabaji, E. Ultrafiltration of highly concentrated monoclonal antibody solutions. Pennsylvania State University the Graduate School College of Engineering, Ph.D. Thesis, pp. 7-170 (2015).
23. Gronemeyer, P., Ditz, R., Strube, J. Trends in upstream and downstream process development for antibody manufacturing. Bioengin. 1, 188-212 (2014).
24. Low, D., O'Leary, R., Pujar, N. S. Future of antibody purification. J. Chromatogr. B 848, 48-63 (2007).
25. Li, Y. Effective strategies for host cell protein clearance in downstream processing of monoclonal antibodies and Fc-fusion proteins. Protein Expression and Purification. 134, 96-103 (2017).
26. Alexander, T. H., Ottens, M. Purifying biopharmaceuticals: knowledge-based chromatographic process development. Trends Biotech. 32, 210-220 (2014).
27. Zhang, K., Liu, X. Mixed-mode chromatography in pharmaceutical and biopharmaceutical applications. J. Pharm. Biomed. Anal. 128, 73-88 (2016).
28. Schuster, S. A., Wagner, B. M., Boyes, B. E., Kirkland, J. J. Wider pore superficially porous particles for peptide separations by HPLC. J. Chromatogr. Sci. 48, 566-571 (2010).
29. Kirkland, J. J, Schuster, S. A., Johnson, W. L., Boyesa, B. E. Fused-core particle technology in high-performance liquid chromatography: An overview. J. Pharmace. Ana. 3, 303-312 (2013).
30. Fernandes, C. S. M. et al. Affitins for protein purification by affinity magnetic fishing. J. Chromatogr. A. 1457, 50-58 (2016).
31. Immunogenicity Assessment for Therapeutic Protein Products https://www.fda.gov/downloads/drugs/guidances/ucm338856.pdf
32. Ramil, F. et al. Elucidation of acid-induced unfolding and aggregation of human immunoglobulin IgG1 and IgG2 Fc. J. Biol. Chem. 287, 1381-1396 (2012).
33. Li, W. et al. Antibody aggregation: insights from sequence and structure. Antibodies 5, 19-40 (2016).
34. Van der Kant, R., et al. Prediction and reduction of the aggregation of monoclonal antibodies. J. Mol. Biol., 429, 1244-1261 (2017).
35. Goswami, S., Wang, W., Arakawa, T., Ohtake, S. Developments and challenges for mAb-based therapeutics. Antibodies 2, 452-500 (2013)
36. Nweke, M. C. Chromatography Resin Characterization to Analyze Lifetime and Performance During Biopharmaceutical Manufacture, A thesis submitted for the degree of Doctor of Philosophy, Department of Biochemical Engineering University College London, (2017).
37. Pinto, N. D., Uplekar, S. D., Moreira, A. R., Rao, G., Frey, D. D. Immunoglobulin G elution in protein A chromatography employing the method of chromatofocusing for reducing the co-elution of impurities. Biotechnol Bioeng. 114, 154-162 (2017).
38. Guiochon, G., Beaver, L. A. Separation science is the key to successful Biopharmaceuticals. J. Chromatogr. A., 1218,8836-8858 (2011).
39. Lowe, C. R., Lowe, A. R, Gupta, G. New developments in affinity chromatography with potential application in the production of biopharmaceutical. J. Biochem. Biophys. Methods 49, 561-574 (2001).
40. FDA approves new eczema drug Dupixent, htTMs://www.fda.gov/NewsEvents/Newsroom/PressAnnouncements/ucm549078.htm.
41. Chiavenna, S. M., Jaworski, J. P. Vendrell, A. State of the art in anti-cancer mAbs. J. Biomed. Sci. 24,15-27 (2017).
42. Peterson, E. C., Gentry, W. B., Owens, S. M. Customizing monoclonal antibodies for the treatment of methamphetamine abuse, current and future applications. Adv. Pharmaco. 69, 107-112 (2014).
43. Bogen, I., Boix, F., Nerem, E., Mørland, J., Andersen, J. M. A Monoclonal antibody specific for 6-monoacetylmorphine reduces acute heroin effects in mice. J. Pharmacol. Exp. Ther. 349, 568-576 (2014).
44. Peterson, E. C., Gentry, W. B., Owens, S. M. Customizing monoclonal antibodies for the treatment of methamphetamine abuse: current and future applications. Adv Pharmacol. 69, 107-127 (2014).
45. Mehta, K. K., Soderquist, R., Shah, P., Marchand, N., Bolton, G. R. Comparing performance of new protein A resins for monoclonal antibody purification. Amer. Pharm. Rev. 21, 61-64 (2018).
46. Gjoka, X., Gantier, R., Schofield, M. Platform for integrated continuous bioprocessing. Biopharm. Intl. 30, 26-32 (2017).
47. Richardson A. Walker J. Continuous solids-discharging centrifugation: a solution to the challenges of clarifying high-cell-density mammalian-cell cultures. Bioproc. Intl. April, 38-47 (2018).
48. Santanal, C. C., Silvajr. I. J., Azevedo, D. C. S., Barretojr, A. B. Aeration, mixing, and hydrodynamics, animal cell bioreactors, in Encyclopedia of Industrial Biotechnology: Bioprocess, Bioseparation, and Cell Technology, edited by Michael C. Flickinger Copyright (John Wiley & Sons, Inc. pp. 1-27, 2010).
49. Gjoka, X., Gantier, R. Schofield, M. Transfer of a three step mAb chromatography process from batch to continuous: Optimizing productivity to minimize consumable requirements. Journal of Biotechnology, 242, 11-18 (2017).
50. Arvedson, T., O'Kelly, J., Yang, B-B. Design rationale and development approach for pegfilgrastim. BioDrugs 29, 185-198 (2015).
51. Kim, A., Sharp, K. A., Honig, B., Electrostatic interactions in macromolecules: theory and applications, Ann. Rev. Biophys. Biophysical Chem. 19, 301-332 (1990).
52. Kramer, R. M., Shende, V. R., Motl, N., Nick, P. C., Scholtz, J. M. Toward a molecular understanding of protein solubility: increased negative surface charge correlates with increased solubility. Biophysical J. 102, 1907-1915 (2012).
53. Atkins, P., de Paula, J., and Friedman. R., Quanta, Matter, and Change: A molecular approach to physical chemistry (W. H. Freeman and Company, New York, N.Y., p. 557. 2009).
54. Paul, A. J., Protein Aggregation during Bioprocessing of Monoclonal Antibodies in Mammalian Cell Culture, Ph.D. Thesis, Universität Ulm, 2016.
55. Potty, A. S. R., Xenopoulos, A. Stress-induced antibody aggregates. BioProcess Intel. 11, 44-52 (2013).
56. Cheung, C. S. F., et al. A new approach to quantification of mAb aggregates using peptide affinity probes. Sci. Rep. 7, Article Number 42497 (2017).
57. Lorber, B., Fischer, F., Bailly, M., Roy, H., Kern, D. Protein Analysis by Dynamic Light Scattering, Methods and Techniques for Students, Biochemistry and Molecular Biology Education, 40,372-382 (2012).
58. Salehi, Z., Najafi, M., RNA preservation and stabilization, Biochem Physiol. 3, 126-129, (2014).
59. Allewell, N. M., Sama, A., The effect of ammonium sulfate on the activity of ribonuclease, Biochimica et Biophysica Acta., 341 484-488, (1974).
60. Bückmann, A. F. Morr, M. Functionalization of poly(ethylene glycol) and monomethoxy-poly(ethylene glycol). Makromol. Chem. 182, 1379-1384 (1981).

We claim:

1. A recognition macromolecule (PEG-RM) composed of a PEGylated recognition macromolecule (PEG-RM) and a target molecule (TM) in which the recognition molecule (RM) is covalently attached to a high molecular weight polyethylene glycol and TM is absorbed to PEG-RM by fundamental interactions including electrostatic (ionic and H-bonding, van der Waals (dipole-dipole), hydrophobic and p interactions, which afford high specificity and binding between PEG-RM and TM and where RM and TM can each be a protein, a nucleic acid or a small molecule and permutations thereof.

2. A product of claim 1 in which the recognition molecule (RM) is a small molecule.

3. A product of claim 1 in which the recognition molecule (RM) is a protein.

4. A product of claim 1 in which the recognition molecule (RM) is a nucleic acid.

5. A product of claim 1 in which the recognition molecule (TM) is a small molecule.

6. A product of claim 1 in which the recognition molecule (TM) is a protein.

7. A product of claim 1 in which the recognition molecule (TM) is a nucleic acid.

8. A process of formation of the macromolecular complex (PEG-RM. TM) in homogeneous media in which the recognition macromolecule (PEG-RM) is added to a biological medium such as blood serum, cell cytoplasm or nucleoplasm or mitochondrial matrix which contains the target molecule (TM).

9. A process of formation of a macromolecular complex (PEG-RM. TG) in which the recognition molecule (RM) is a small molecule, a protein or a nucleic acid and the target molecule (TM) can be a small molecule, a protein or a nucleic acid and the resulting nine permutations thereof.

10. A process of salt-assisted phase transition of a macromolecular complex (PEG-RM. TM) in which the water soluble macromolecular complex (PBG-RM.TM) separates as a solid or a semisolid from a biological medium such as blood serum, cell cytoplasm or nucleoplasm or mitochondrial matrix by the addition of a minimum quantity of salts, preferably ammonium sulfate.

11. A process of separating the solid or a semisolid macromolecular complex of claim 10 (PEG-RM.TM) by filtration.

12. A process of separating the solid or a semisolid macromolecule complex of claim 10 (PEG-RM.TM) by or centrifugation.

13. A process of convening the macromolecular complex (PEG-RM.TM) to its components (PBG-RM+TM) by firstly dissolving it in an appropriate buffer, preferably Tris buffer, and secondly reducing fee pH of the buffer.

14. A process of con veiling the macromolecular complex (PEG-RM.TM) to its components (PEG-RM+TM) by firstly dissolving it in an appropriate buffer, preferably Tris buffer, and secondly Increasing the ionic strength of the buffer by the addition of an appropriate quantity of salt.

15. A process of converting the recognition macromolecule (PEG-RM) from solution to a semisolid by the addition of a minimum quantity of salt, preferably ammonium sulfate.

16. A process of separating the semisolid recognition macromolecule (PEG-RM) by filtration and obtaining the pure or highly pure target (TM) molecule in solution.

17. A process of separating the semisolid recognition macromolecular (PEG-KM) by centrifugation and obtaining the pure or highly pure target (TM) molecule in solution

18. A homogenous process for separation of target molecules (TM) in which no leaching of the recognition molecule (RM) is observed.

19. A homogenous process for separation of proteins as target molecules (TM) in which no aggregate of the target molecule is observed

20. (canceled)

21. (canceled)

22. (canceled)
